(19) United States
(12) Patent Application Publication
(10) Pub. No.: US 2026/0191769 A1
(43) Pub. Date: Jul. 9, 2026

(54) DRUG ELUTING IMPLANTS AND USES THEREOF

(71) Applicant: MUSC FOUNDATION FOR RESEARCH DEVELOPMENT, Charleston, SC (US)

(72) Inventors: Josef Eichinger, Charleston, SC (US); Michael Yost, Charleston, SC (US); Sarah Grace Dennis-Little, Charleston, SC (US); Michael Gerard Schmidt, Charleston, SC (US)

(21) Appl. No.: 19/133,166

(22) PCT Filed: Nov. 28, 2023
(86) PCT No.: PCT/US2023/081370

§ 371 Date: May 27, 2025

Related U.S. Application Data
(60) Provisional application No. 63/385,102, filed on Nov. 28, 2022.

(51) Int. Cl. A61K 9/00; A61K 9/06; A61K 38/14; A61K 47/36; A61K 47/42

(52) U.S. Cl. CPC A61K 9/0024; A61K 9/06; A61K 38/14; A61K 47/36; A61K 47/42

(57) ABSTRACT

Described herein are exemplary embodiments of implantable therapeutic and/or preventative delivering devices that can be composed of one or more layers of a collage-alginate hydrogel and uses thereof.

### CROSS-REFERENCE TO RELATED APPLICATIONS

This application claims the benefit of and priority to U.S. Provisional Patent Application No. 63/385,102, filed on Nov. 28, 2022, entitled “Drug Eluting Implants and Uses Thereof,” the contents of which is incorporated by reference herein in its entirety.

### TECHNICAL FIELD

The subject matter disclosed herein is generally directed to compositions, materials, and methods for delivering compounds, such as therapeutic compounds, particularly over an extended period of time.

### BACKGROUND

Surgical site infections remain a significant concern after surgeries, such as orthopedic procedures, particularly joint replacement and other prosthetic placements. There is a paucity of commercially available devices, preventions, and/or therapeutics for sustained delivery of antibiotics in orthopedic, surgical, and other applications where internal long-term and/or reemergent infections are an issue.

Citation or identification of any document in this application is not an admission that such a document is available as prior art to the present invention.

### SUMMARY

In some aspects, the devices and techniques described herein relate to an implantable device including: one or more layers of a formable gel, the formable gel of each of the one or more layers including: an amount of collagen; an amount of alginate; wherein the collagen and the alginate form a formable gel capable of implantation; and one or more therapeutic and/or preventative compounds dispersed throughout the formable gel, wherein the formable gel is configured to release the one or more therapeutic and/or preventative compounds over a period of time.

In some aspects, the devices and techniques described herein relate to an implantable device, wherein the amount of collagen and/or the amount of alginate is/are effective amounts.

In some aspects, the devices and techniques described herein relate to an implantable device, wherein a ratio of collagen to alginate is an effective ratio.

In some aspects, the devices and techniques described herein relate to an implantable device, wherein the alginate, the collagen, or both are modified.

In some aspects, the devices and techniques described herein relate to an implantable device, wherein the alginate is at least partially oxidized.

In some aspects, the devices and techniques described herein relate to an implantable device, wherein the alginate is about 1-50%, about 3-30%, about 5-30%, about 7-30%, about 10-30%, about 12-30%, about 15-30%, about 20-30%, about 25-30%, about 1-25%, about 3-25%, about 5-25%, about 7-25%, about 10-25%, about 12-25%, about 15-25%, about 20-25%, about 1-20%, about 3-20%, about 5-20%, about 7-20%, about 10-20%, about 12-20%, about 15-20%, about 1-15%, about 3-15%, about 5-15%, about 7-15%, about 10-15%, about 12-15%, about 1-12%, about 1-12%, about 5-12%, about 7-12%, about 10-12%, about 1-10%, 1-3%, about 5-10%, about 7-10%, about 1-7%, about 3-7%, about 5-7%, about 1-5%, about 3-5%, about 1-3% 3-50%, about 5-50%, about 7-50%, about 10-50%, about 12-50%, about 15-50%, about 20-50%, about 25-50%, about 30-50%, about 35-50%, about 40-50%, about 45-50%, about 30-45%, about 30-40%, about 30-35%, about 25-45%, about 25-40%, about 25-35%, about 20-45%, about 20-40%, about 20-35%, about 15-45%, about 15-40%, about 15-35%, about 10-45%, about 10-40%, about 10-35%, about 5-45%, about 5-40%, about 1-35%, about 1-45%, about 1-40%, or about 1-35% oxidized.

In some aspects, the devices and techniques described herein relate to an implantable device, wherein the period of time is at least 1-42 days or more, about 7-42 days, about 14-42 days, about 21-42 days, about 28-42 days, about 1-28 days, about 7-28 days, about 14-28 days, about 21-28 days, about 1-21 days, about 7-21 days, about 14-21 days, about 1-14 days, about 7-14 days, or about 1-7 days.

In some aspects, the devices and techniques described herein relate to an implantable device, wherein one or more layers of the formable gel is substantially homogenous.

In some aspects, the devices and techniques described herein relate to an implantable device, wherein one or more layers of the formable gel is heterogenous.

In some aspects, the devices and techniques described herein relate to an implantable device, wherein the implantable device further includes one or more impermeable but degradable layers, wherein each of the one or more impermeable but degradable layers is sandwiched between two layers of the formable gel and/or is forms an external layer surrounding the one or more formable gel layers and/or one or more other degradable layers.

In some aspects, the devices and techniques described herein relate to an implantable device, wherein one or more of the one or more impermeable but degradable layers is (a) biodegradable, (b) degradable in response to an external stimuli, or both (a) and (b).

In some aspects, the devices and techniques described herein relate to an implantable device, wherein one or more therapeutic and/or preventative compounds are each present in one or more of the one or more layers of the formable gel at 1-70 wt. % of the formable gel layer, at 1-65% of the formable gel layer, 1-60% of the formable gel layer, 1-55% of the formable gel layer, 1-50% of the formable gel layer, 1-45 wt. % of the formable gel layer, 1-40 wt. % of the formable gel layer, 1-35 wt. % of the formable gel layer, 1-30 wt. % of the formable gel layer, 1-25 wt. % of the formable gel layer, 1-20 wt. % of the formable gel layer, 1-15 wt. % of the formable gel layer, 1-10 wt. % of the formable gel layer, or 1-5 wt. % of the formable gel layer.

In some aspects, the devices and techniques described herein relate to an implantable device, wherein the one or more therapeutic and/or preventative compounds are selected from the group consisting of: an anti-infective, a chemotherapeutic, an immunomodulator, an antipyretic, an analgesic, an antispasmodic, an anti-inflammatory, an anti-histamine, a radiation sensitizer, a chemotherapeutic sensitizer, an anti-infective sensitizer, a biologic factor, a bisphosphonate, or any combination thereof.

In some aspects, the devices and techniques described herein relate to an implantable device, wherein the one or more therapeutic and/or preventative compounds includes an anti-infective agent.

In some aspects, the devices and techniques described herein relate to an implantable device, wherein the one or more therapeutic and/or preventative compounds include an antibiotic.

In some aspects, the devices and techniques described herein relate to an implantable device, wherein the one or more therapeutic and/or preventative compounds includes an aminoglycoside or a derivative thereof.

In some aspects, the devices and techniques described herein relate to an implantable device, wherein the aminoglycoside is selected from paromomycin, tobramycin, gentamicin, amikacin, kanamycin, neomycin, and any combination thereof.

In some aspects, the devices and techniques described herein relate to an implantable device, wherein the one or more therapeutic and/or preventative compounds are each independently selected from an aminoglycoside or a derivative thereof, a carbapenem or a derivative thereof, a cephalosporin or a derivative thereof, a glycopeptide or a derivative thereof, a glycylcycline or a derivative thereof, a lincomycin or a derivative thereof, a macrolide or a derivative thereof, a penicillin or a derivative thereof, a quinolone or a derivative thereof, a sulfonamide or a derivative thereof, a tetracycline or a derivative thereof, or any combination thereof.

In some aspects, the devices and techniques described herein relate to an implantable device, wherein (a) the aminoglycoside includes paromomycin, tobramycin, gentamicin, amikacin, kanamycin, and neomycin or any combination thereof; (b) the carbapenem includes doripenem, meropenem, ertapenem, and cilastatin/imipenem; (c) the cephalosporin includes cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, ceftazidime, or any combination thereof; (d) the glycopeptide includes vancomycin, dalbavancin, oritavancin, telvancin, or any combination thereof; (e) the glycylcycline includes tigecycline; (f) the lincomycin includes clindamycin or lincomycin; (g) the macrolide includes telithromycin, fidaxomicin, erythromycin, azithromycin, clarithromycin, dirithromycin, troleandomycin, linezolid, sulfamethoxazole/trimethoprim, rifaximin, chloramphenicol, fosfomycin, metronidazole, aztreonam, bacitracin, or any combination thereof; (h) the penicillin includes amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, nafcillin, or any combination thereof; (i) the quinolone includes lomefloxacin, norfloxacin, ofloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, sparfloxacin, or any combination thereof; (j) the sulfonamide includes sulfamethoxazole/trimethoprim, sulfasalazine, sulfasoxazole, or any combination thereof; (k) the tetracycline (e.g. demeclocycline, minocycline, doxycycline/salicyclic acid, doxycycline/omega-3 polyunsaturated fatty acids, and tetracycline; or (1) any combination of (a)-(k).

In some aspects, the devices and techniques described herein relate to an implantable device, wherein the implantable device is effective to treat and/or prevent microbial infection, microbial proliferation, biofilm formation, or any combination thereof.

Described in some aspects herein are kits that include the implantable device of the present disclosure.

Described in some aspects herein are methods that relate to inserting or otherwise delivering an implantable device according to the present disclosure into a surgical site or wound in a subject in need thereof.

In some aspects, the techniques described herein relate to a method, wherein the surgical site or wound is in or near a joint of the subject, at or near an implanted foreign object in a subject, at or near the site of a skin or other soft tissue graft.

In some aspects, the techniques described herein relate to a method, wherein the subject has or is at risk for developing a surgical site, implant foreign object site, or wound site infection.

In some aspects, the techniques described herein relate to a method, wherein the subject has or is at risk for developing a microbial infection.

In some aspects, the techniques described herein relate to a method, wherein the microbial infection includes a bacterial infection, a yeast infection, or both.

In some aspects, the techniques described herein relate to a method, wherein the bacterial infection is a chronic bacterial infection, a resistant bacterial infection, or both.

In some aspects, the techniques described herein relate to a method, wherein the bacterial infection includes a biofilm.

In some aspects, the techniques described herein relate to a method, wherein the bacterial infection is caused by a bacteria of the species Staphylococcus, Streptococcus, Pseudomonas, Enterococcus, Escherichia, Acinetobacter, or any combination thereof.

In some aspects, the techniques described herein relate to a method, wherein the bacterial infection is caused by Staphylococcus aureus.

In some aspects, the techniques described herein relate to a method, wherein implanting occurs during a primary surgical procedure performed on the subject.

In some aspects, the techniques described herein relate to a method, wherein implanting occurs during a revision surgical procedure performed on the subject.

In some aspects, the techniques described herein relate to a method, wherein implanting occurs within 0.5-72 hours of a non-surgical wound causing trauma to the subject.

In some aspects, the techniques described herein relate to a method, wherein the wound is a burn or other trauma requiring a skin graft.

In some aspects, the techniques described herein relate to a method, wherein the implanted foreign object is a penile implant, a soft tissue implant, a replacement joint or portion thereof, a fixation implant, or any combination thereof.

In some aspects, the techniques described herein relate to a method, wherein (a) the soft tissue implant includes a breast implant, calf implant, buttock implant, or a cheek implant; (b) the fixation implant includes a screw, a plate, a cage, a rod, a pin, an anchor, a disc, or any combination thereof; or (c) both (a) and (b).

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows images of gels loaded with a 33 wt % concentration of vancomycin after 14 days. PBS (Phosphate Buffered Saline) at 37° C. to replicate body fluid and was combined with the gel and PBS in a well in a tissue culture plate.

FIG. 2 shows a table of ELISA results from an antibiotic elution trail from collagen-alginate gels. These results are graphically represented in FIG. 3.

FIG. 3 shows a graph demonstrating ELISA results set forth in FIG. 2. x-axis vancomycin concentration and y axis regular and cross linked gels.

FIG. 4 shows a table with results from an evaluation of the degradation kinetics of collagen-alginate gels over 28 days in vitro. Two different concentrations of gels with and without UV crosslinking were fabricated and tested. Samples with either vancomycin or penicillin were placed in simulated body fluid, with and without the addition of collagenase to mimic the in vivo environment. At 7, 14, 28, 42 days, samples were dried, weighed and the degradation rate was calculated.

FIG. 5 shows an image of dried gels at days 7 and 14. The “C” denotes that the gel in the container was crosslinked.

FIG. 6 shows an exemplary application of the gels, specifically of it being placed in a shoulder joint during an orthopedic procedure.

FIGS. 7A-7B show images of zone of inhibition studies of various eluting gels for MRSA, which is notably highly resistant to a variety of antibiotics. The center disk in each plate is a Vancomycin 30 mcg control, the disk at the top (at the 12 o'clock position) is Vancomycin 5 mcg control. Going clockwise following the Vancomycin 5 mcg control are the following days 3, 7, 14, 21, and 28. FIG. 7A shows collagen and FIG. 7B shows peroxide.

FIG. 8 shows a graph demonstrating the effect of gel oxidation on vancomycin release from the gel.

FIGS. 9A-9B show exemplary embodiments of the collagen-alginate gels described herein. In some embodiments, the gel is homogenous (FIG. 9A). In some embodiments, the gel can contain 2 or more different layers of collagen-alginate-antibiotic and can optionally include one or more impermeable and/or biodegradable layers (FIG. 9B).

FIG. 10 can demonstrate that selectively oxidized collagen-alginate hydrogels continuously release therapeutic concentrations of an antibiotic. The far-left panel is a graph of the ELISA results for vancomycin released into the simulated body fluid vs time. These data show that the release characteristics of the drug can be controlled by manipulation of the oxidation state of the hydrogel. As can be seen from the graph, therapeutic levels of antibiotic are still present after 14 days. Inset: A photograph of the ABED gels.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours, days, weeks, to months.

As used herein, “chemotherapeutic agent” or “chemotherapeutic” refers to a therapeutic agent utilized to prevent or treat cancer.

As used herein, “dose,” “unit dose,” or “dosage” can refer to physically discrete units suitable for use in a subject, each unit containing a predetermined quantity of the implantable device described herein thereof calculated to produce the desired response or responses in association with its administration.

As used herein, “hydrogel” refers to a gelatinous colloid, or aggregate of polymeric molecules in a finely dispersed semi-solid state, where the polymeric molecules are in the external or dispersion phase and water (or an aqueous solution) is forms the internal or dispersed phase. Generally, hydrogels are at least 90% by weight of an aqueous solution but can be different as described elsewhere herein.

As used herein, “immunomodulator,” refers to an agent, such as a therapeutic agent, which is capable of modulating or regulating one or more immune function or response.

As used herein, “infection” as used herein refers to presence of an infective agent, such as a pathogen, e.g., a microorganism, in or on a subject, which, if its presence or growth were inhibited, would result in a benefit to the subject. Hence, the term refers to the state produced by the establishment, more particularly invasion and multiplication, of an infective agent, such as a pathogen, e.g., a microorganism, in or on a suitable host. An infection may produce tissue injury and progress to overt disease through a variety of cellular and toxic mechanisms.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

As used herein, “preventative” and “prevent” refers to hindering or stopping a disease or condition before it occurs, even if undiagnosed, or while the disease or condition is still in the sub-clinical phase.

As used herein, the term “radiation sensitizer” refers to agents that can selectively enhance the cell killing from irradiation in a desired cell population, such as tumor cells, while exhibiting no single agent toxicity on tumor or normal cells.

As used herein, “substantial” and “substantially,” specify an amount of between 95% and 100%, inclusive, between 96% and 100%, inclusive, between 97% and 100%, inclusive, between 98% 100%, inclusive, or between 99% 100%, inclusive.

As used interchangeably herein, the terms “sufficient” and “effective,” can refer to an amount (e.g., mass, volume, dosage, concentration, and/or time period) needed to achieve one or more desired result(s). For example, a therapeutically effective amount refers to an amount needed to achieve one or more therapeutic or preventive effects or other desired effect. In the context of the hydrogels herein, an effective amount (or ratio) of collagen and/or alginate refers to the amount of each (or the ratio of the two), when considered in context of the rest of the hydrogel components that is effective to provide a formable hydrogel that can have a desired release profile, such as release a therapeutically effective and/or preventative amount of a therapeutic or preventive agent contained in said hydrogel layer over a desired period of time such as 1-42 days or other period of time set forth elsewhere herein. In connection with a therapeutic and/or preventive amount of a therapeutic or preventative agent, the amount can be a minimum effective amount (or concentration). A minimum effective amount (or concentration) is the least effective amount/concentration needed to achieve a therapeutic and/or preventive effect. One of ordinary skill in the art will appreciate the desired therapeutic and/or preventive effect based on the therapeutic and/or preventive included in the implantable device. For example, where an antibiotic is included, the effective amount will be the amount or least amount needed to kill and/or inhibit the growth and/or proliferation of one or more bacteria.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Surgical site infections remain a significant concern after surgeries, such as orthopedic and cosmetic procedures, particularly joint replacement and other prosthetic or cosmetic implant placements, during the treatment of chronic and other wounds, and treatment of burns. There is a paucity of commercially available devices, preventions, and/or therapeutics for sustained delivery of antibiotics in orthopedic, surgical, and other applications where internal long-term and/or reemergent infections are an issue.

With that said, several exemplary embodiments disclosed herein provide implantable devices that contain one or more layers of a formable gel that can each contain a therapeutic and/or preventative compound and be capable of releasing the therapeutic and/or preventative compound(s) into the environment surrounding the implantable device over a period of time, particularly over an extended period of time (e.g., weeks to months or more). The implantable devices can be included in kits, such as with a delivery device, storage containers, and/or solutions for delivery and/or storage. Several exemplary embodiments described herein provide methods of implanting the implantable devices described herein into a wound, such as a surgical site or surgical wound or other wound caused by trauma or is secondary to another condition (e.g., such as ulcers, necrosis etc.) in a subject. In some embodiments, the method can provide a treatment or prevention of a surgical site infection, foreign implant site infection, skin or other soft tissue graft infection, and/or wound infection, including but not limited to chronic wounds and/or those covering broad portions of the body or other hard to heal wounds. In some cases, the wound is one that requires a skin or other soft tissue graft. The implantable devices described herein can be effect to treat and/or prevent microbial proliferation and/or biofilm formation.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Drug Eluting Implantable Devices

Described in several exemplary embodiments herein are implantable devices that contain or are entirely composed of one or more layers of a formable gel, the formable gel of each of the one or more layers contains or is entirely composed of an amount of collagen; an amount of alginate, where the collagen and the alginate form a formable gel capable of implantation; and one or more therapeutic and/or preventative compounds dispersed throughout the formable gel, where the formable gel is configured to release the one or more therapeutic and/or preventative compounds over a period of time. The formable gel can be a hydrogel in which the matrix portion is formed from collagen and alginate.

In some embodiments, the total weight of the implantable device is about 1 to/or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μg, mg, or g or be any numerical value with any of these ranges when weighed, for example, when fully hydrated or substantially undegraded. In some embodiments, the total volume of the implantable device is about 1 to/or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 nL, μL, or mL (or cc) or be any numerical value with any of these ranges when, for example, fully hydrated or substantially undegraded.

In some embodiments, the total weight of each formable gel layer present in the implantable device are each independently about 1 to/or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 ng, μg, mg, or g or be any numerical value with any of these ranges when weighed, for example, when fully hydrated or substantially undegraded. In some embodiments, the total volume of each formable gel layer present in the implantable device are each independently about 1 to/or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 μL or mL (or cc) or be any numerical value with any of these ranges when, for example, fully hydrated or substantially undegraded.

The amount of collagen present in each formable gel layer of the implantable device can each independently be about 0.1, to/or about 0.2, 0.3, 0.4, 0.5, 0.6, 0.7, 0.8, 0.9, 1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, w/v, or v/v or any value or range of values therein of the total amount of the formable gel layer.

In some embodiments, the amount of collagen included each formable gel layer in the implantable device is each independently about 1 to/or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 ng, μg, mg, or g.

The amount of alginate present in each formable gel layer of the implantable device can each independently be about 0.1, to/or about 0.2, 0.3, 0.4, 0.5, 0.6, 0.7, 0.8, 0.9, 1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, w/v, or v/v or any value or range of values therein of the total amount of the formable gel layer.

In some embodiments, the amount of alginate included each formable gel layer in the implantable device is each independently about 1 to/or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 ng, μg, mg, or g.

In some embodiments, the alginate and collagen together make up about 0.1, 0.2, 0.3, 0.4, 0.5, 0.6, 0.7, 0.8, 0.9, 1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, to/or about 99.9% w/w, w/v, or v/v or any value or range of values therein of the total amount of the formable gel layer.

The collagen and alginate can be present in the formable gel layer of the implantable device at a ratio relative to each other. In some embodiments, the ratio of collagen to alginate is 0.1-10:1, 1:0.1-10, 0.1-10:0.1-10. In some embodiments the ratio of collagen to alginate is 1:3, 1:2.75; 1:2.5, 1:2.25; 1:2; 1:1.75; 1:1.5. 1:1.25, 1:1, 1.25:1, 1.5:1, 1.75:1, 2:1, 2.25:1, 2.5:1, 2.75:1 or 3:1.

In some embodiments, the amount of collagen and/or the amount of alginate is/are effective amounts. In some embodiments, the ratio of collagen to alginate is an effective ratio.

In some embodiments, the alginate, the collagen, or both are modified. In some embodiments the modification is oxidation In some embodiments the modification is acidification. In some embodiments the modification is heating. In some embodiments, the alginate is at least partially oxidized. In some embodiments the collagen is acidified. In some embodiments the collagen is acidified to between a pH of 1-6, or 2-5, or 3-4. In some embodiments the collagen is acidified to a pH of 3.0, 3.1, 3.2, 3.3, 3.4, 3.5, 3.6, 3.7, 3.8, 3.9 or 4.0. In some embodiments the collagen is heated for at least 1, 2, 3, 4, 5, 6, 7, 8, 9, 10, 11 or 12 hours.

In some embodiments, the alginate is about 1-50% oxidized. In some embodiments, the alginate is about 1% to/or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50% oxidized. In some embodiments, the alginate is about 3-50%, about 5-50%, about 7-50%, about 10-50%, about 12-50%, about 15-50%, about 20-50%, about 25-50%, about 30-50%, about 35-50%, about 40-50%, about 45-50%, about 30-45%, about 30-40%, about 30-35%, about 25-45%, about 25-40%, about 25-35%, about 20-45%, about 20-40%, about 20-35%, about 15-45%, about 15-40%, about 15-35%, about 10-45%, about 10-40%, about 10-35%, about 5-45%, about 5-40%, about 1-35%, about 1-45%, about 1-40%, or about 1-35% oxidized In some embodiments, the alginate is 1-30% oxidized. In some embodiments the alginate is about 3-30%, about 5-30%, about 7-30%, about 10-30%, about 12-30%, about 15-30%, about 20-30%, about 25-30%, about 1-25%, about 3-25%, about 5-25%, about 7-25%, about 10-25%, about 12-25%, about 15-25%, about 20-25%, about 1-20%, about 3-20%, about 5-20%, about 7-20%, about 10-20%, about 12-20%, about 15-20%, about 1-15%, about 3-15%, about 5-15%, about 7-15%, about 10-15%, about 12-15%, about 1-12%, about 1-12%, about 5-12%, about 7-12%, about 10-12%, about 1-10%, 1-3%, about 5-10%, about 7-10%, about 1-7%, about 3-7%, about 5-7%, about 1-5%, about 3-5%, or about 1-3% oxidized.

The collagen can be any suitable collagen. In some embodiments, the collagen is a type I, type II, type III, type IV collagen, or any combination thereof. In some embodiments, the collagen is a type I collagen. In some embodiments, the collagen is a type III collagen.

Each layer can include additional components such as salts (can be in the form of a saline solution or other buffered salt solution).

The pH of each formable gel layer can independently be acidic, neutral, or basic. In some embodiments, the pH of each formable gel layer is each independently about 7 to about 7.4, such as about 6.5, 6.6, 6.7, 6.8, 6.9, 7.0, 7.1, 7.2, 7.3, 7.4, 7.5, 7.6, 7.7, 7.8, 7.9, or 8.0. In some embodiments, the pH of each formable gel layer is about the same pH. In some embodiments, the pH of at least two formable gel layers are different. In some embodiments, the pH of at least two formable gel layers are the same. In some embodiments, the pH of each of the formable gel layers is different.

As is discussed elsewhere herein, current eluting gels fail to allow for extended periods of drug elution period of time. In some embodiments, the implantable device can provide drug elution for extending periods of time, such as those of 14 days or longer (e.g., 14-28, 36, 42, or 48 days or more. In some embodiments the period of time is at least 1-42 days or more, about 7-42 days, about 14-42 days, about 21-42 days, about 28-42 days, about 1-28 days, about 7-28 days, about 14-28 days, about 21-28 days, about 1-21 days, about 7-21 days, about 14-21 days, about 1-14 days, about 7-14 days, or about 1-7 days. In some embodiments, the period of time the implantable device releases a therapeutic and/or preventative compound is about 1 to/or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or more days.

In some embodiments, one or more layers of the formable gel is substantially homogenous.

In some embodiments, one or more layers of the formable gel is heterogenous. In this context, “heterogenous” refers to a difference in at least one characteristic (e.g., hydrogel composition (e.g., amount of alginate and/or collagen, amount of therapeutic and/or preventative compound(s), etc.) throughout the gel, pH, and/or other characteristic. For example, a layer can be formed such that a concentration gradient or different concentrations of the therapeutic and/or preventative exists in the layer. This can allow for fine tuning of a release profile. For example, in embodiments, where pockets or strips of therapeutic and/or preventative compounds are present can provide for bolus or pulsatile release from that layer as the gel degrades.

In some embodiments where multiple layers are included in the implantable device, two or more of the layers are the same. In some embodiments where multiple layers are included in the implantable device, all of the layers can be the same. In some embodiments where multiple layers are included in the implantable device, two or more of the layers are different in at least one characteristic. In some embodiments where multiple layers are included in the implantable device, all of the layers are different from each other in at least one characteristic. In some embodiments, the difference is the type of therapeutic and/or preventative compound(s) included in the layer, the amount of collagen and/or alginate, the percent of alginate oxidation, or any combination thereof.

In some embodiments, the implantable device contains one or more impermeable but degradable layers. In some embodiments each of the one or more impermeable but degradable layers is sandwiched between two layers of the formable gel and/or forms an external layer surrounding the one or more formable gel layers and/or one or more other degradable layers.

In some embodiments, one or more of the one or more impermeable but degradable layers is biodegradable and/or is degradable in response to an external stimuli. Exemplary external stimuli include, but are not limited to, pH, light energy, electromagnetic energy, magnetic energy, and acoustic energy. In this way release can be controlled externally, such as by applying a light source (e.g., cold laser) to the skin of a subject over where the implant is to stimulate release of a therapeutic and/or preventative within the layer.

In some embodiments where the implantable device has multiple formable gel layers and, optionally, one or more impermeable but degradable layers, the device is configured as a core surrounded by laminar layers disposed about the core layer such that if the device were cut in half, each half would be the same from the core out. The implantable device can have any three-dimensional regular or irregular shape. The implantable device can have any width, length, or thickness. In some embodiments, the largest dimension (width, length, height, diameter etc.) is 0.001 to 1 nm, micrometer, or centimeter.

Each layer can contain one or more (e.g., 1, 2, 3, 4, 5 or more therapeutic and/or preventative compounds (also referred to herein as agents). In some embodiments, the one or more therapeutic and/or preventative compounds are each present in one or more of the one or more layers of the formable gel at 1-70 wt. % of the formable gel layer. n some embodiments, the one or more therapeutic and/or preventative compounds are each present in one or more of the one or more layers of the formable gel at 1-65% of the formable gel layer, 1-60% of the formable gel layer, 1-55% of the formable gel layer, 1-50% of the formable gel layer, 1-45 wt. % of the formable gel layer, 1-40 wt. % of the formable gel layer, 1-35 wt. % of the formable gel layer, 1-30 wt. % of the formable gel layer, 1-25 wt. % of the formable gel layer, 1-20 wt. % of the formable gel layer, 1-15 wt. % of the formable gel layer, 1-10 wt. % of the formable gel layer, or 1-5 wt. % of the formable gel layer. In some embodiments, the one or more therapeutic and/or preventative compounds are each present in one or more of the one or more layers of the formable gel at 1, to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 wt. % of the formable gel layer.

In some embodiments, each layer independently contains about 1 to/or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 μg, ng, μg, mg, or g of each of the one or more therapeutic and/or preventive compounds.

In some embodiments, each layer independently contains about 1 to/or 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M of each of the one or more therapeutic and/or preventive compounds.

In some embodiments, each layer independently contains about 1 to/or 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU of each of the one or more therapeutic and/or preventive compounds.

In some embodiments, the one or more therapeutic and/or preventative compounds are selected from the group of: an anti-infective, a chemotherapeutic, an immunomodulator, an antipyretic, an analgesic, an antispasmodic, an anti-inflammatory, an anti-histamine, a radiation sensitizer, a chemotherapeutic sensitizer, an anti-infective sensitizer, a biologic factor, a bisphosphonate, or and combinations thereof.

Suitable immunomodulators include, but are not limited to, prednisone, azathioprine, 6-MP, cyclosporine, tacrolimus, methotrexate, interleukins (e.g., IL-2, IL-7, and IL-12), cytokines (e.g. interferons (e.g. IFN-a, IFN-β, IFN-ç, IFN-K, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable antipyretics include, but are not limited to, non-steroidal anti-inflammatory agents (e.g., ibuprofen, naproxen, ketoprofen, and nimesulide), aspirin and related salicylates (e.g. choline salicylate, magnesium salicylae, and sodium salicylate), paracetamol/acetaminophen, metamizole, nabumetone, phenazone, and quinine.

Suitable analgesics include, but are not limited to, paracetamol/acetaminophen, nonsteroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylate, and sodium salicylate).

Suitable antispasmodics include, but are not limited to, mebeverine, papverine, cyclobenzaprine, carisoprodol, orphenadrine, tizanidine, metaxalone, methocarbamol, chlorzoxazone, baclofen, dantrolene, baclofen, tizanidine, and dantrolene. Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H1-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g. cimetidine, famotidine, lafutidine, nizatidine, rafitidine, and roxatidine), tritoqualine, catechin, cromoglicate, nedocromil, and p2-adrenergic agonists.

Suitable anti-infectives include, but are not limited to, amebicides (e.g. nitazoxanide, paromomycin, metronidazole, tinidazole, chloroquine, miltefosine, amphotericin b, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), quinolones (e.g. lomefloxacin, norfloxacin, ofloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicylic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

Suitable radiation sensitizers include, but are not limited to, 5-fluorouracil, platinum analogs (e.g. cisplatin, carboplatin, and oxaliplatin), gemcitabine, DNA topoisomerase I-targeting drugs (e.g. camptothecin derivatives (e.g. topotecan and irinotecan)), epidermal growth factor receptor blockade family agents (e.g. cetuximab, gefitinib), farnesyltransferase inhibitors (e.g., L-778-123), COX-2 inhibitors (e.g. rofecoxib, celecoxib, and etoricoxib), bFGF and VEGF targeting agents (e.g. bevazucimab and thalidomide), NBTXR3, Nimoral, trans sodium crocetinate, NVX-108, and combinations thereof. See also e.g., Kvols, L. K., J Nucl Med 2005; 46:187S-190S.

Suitable bioactive factors include those that promote soft tissue and/or bone growth, remodeling, regeneration, and/or healing. Such bioactive factors include, but are not limited to, growth factors (e.g., TGF-beta, fibroblast growth factors (FGFs) (e.g., basic FGF), and Insulin like growth factors (IGFs) (e.g., IGF-I), Vascular endothelial growth factor (VEGF), and platelet-derived growth factor (PDGF)), hormones, fatty acids, bone morphogenetic proteins (BMPs) (e.g., BMP 2, BMP3, BMP4, BMP6, and BMP7), and any combination thereof.

In some embodiments, the one or more therapeutic and/or preventative compounds comprise at least an anti-infective agent. In some embodiments, the one or more therapeutic and/or preventative compounds comprise at least an antibiotic. In some embodiments, the one or more therapeutic and/or preventative compounds comprise an aminoglycoside or a derivative thereof (e.g. paromomycin, tobramycin, gentamicin, amikacin, kanamycin, and neomycin), a carbapenem or a derivative thereof (e.g. doripenem, meropenem, ertapenem, and cilastatin/imipenem), a cephalosporin or a derivative thereof (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), a glycopeptide or a derivative thereof (e.g. vancomycin, dalbavancin, oritavancin, and telvancin), glycylcyclines (e.g. tigecycline), a lincomycin or a derivative thereof (e.g. clindamycin and lincomycin), a macrolide or a derivative thereof (e.g. telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin), a penicillin (e.g., amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), a quinolone (e.g. lomefloxacin, norfloxacin, ofloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), a sulfonamide (e.g. sulfamethoxazole/trimethoprim, sulfasalazine, and sulfasoxazole), a tetracycline (e.g. doxycycline, demeclocycline, minocycline, doxycycline/salicylic acid, doxycycline/omega-3 polyunsaturated fatty acids, and tetracycline), or a combination thereof.

In some embodiments, the therapeutic and/or preventive compound is effective against a bacteria of the species Staphylococcus, Streptococcus, Pseudomonas, Enterococcus, Escherichia, Acinetobacter, or any combination thereof. In some embodiments, the therapeutic and/or preventive compound is effective against Staphylococcus aureus. In some embodiments, the therapeutic and/or preventive compound is effective against Methicillin-resistant Staphylococcus aureus (MRSA).

In some embodiments, the implantable device and/or therapeutic compound thereof is effective to treat and/or prevent biofilm formation, microbial proliferation, or both. In some embodiments, the biofilm formation, microbial proliferation, or both is at a surgical site, closure, or wound (e.g., any wound made to one or more parts of the body during a surgical procedure), near an implanted foreign object in a subject, a non-surgical wound (e.g., one cause by trauma or other condition (e.g., ulceration, necrosis, such as that caused by diabetes or other circulatory conditions), at or near a graft, such as a skin graft or other soft tissue graft (including, allogenic, xenogeneic, autologous grafts), or any combination thereof. In some embodiments, the surgical or non-surgical wound is a dermal wound. Exemplary implantable foreign objects include but are not limited to implantable devices such as catheters, stents, fixation devices (e.g., screws, pins, rods, plates, cages, anchors, discs, balls, sutures, wires, etc.), replacement joints or components thereof, other cosmetic implants, other orthopedic implants, and/or the like. In some embodiments, the foreign object implant is a penile implant, breast implant, buttock implant, cheek implant, lip implant or other cosmetic or orthopedic implant.

Exemplary methods of making hydrogels are described in the Working Examples herein and will be known to one of ordinary skill in the art in view of the description provided herein. Methods of oxidizing alginate are described for example, in the Working Examples herein.

### Kits

Any of the compounds, compositions, formulations, and/or devices described herein or a combination thereof can be presented as a combination kit. As used herein, the terms “combination kit” or “kit of parts” refers to the c compounds, compositions, formulations, and/or devices and any additional components that are used to package, sell, market, deliver, and/or administer the combination of elements or a single element, such as the active ingredient, contained therein. Such additional components include, but are not limited to, packaging, syringes, blister packages, bottles, and the like. The separate kit components can be contained in a single package or in separate packages within the kit.

In some embodiments, the combination kit also includes instructions printed on or otherwise contained in a tangible medium of expression. The instructions can provide information regarding the content of the c compounds, compositions, formulations, and/or devices described herein or a combination thereof contained therein, safety information regarding the content of the compounds, compositions, formulations, and/or devices (e.g., implantable devices, information regarding the dosages, indications for use, and/or recommended treatment regimen(s) for the compounds, compositions, formulations, and/or devices contained therein. In some embodiments, the instructions can provide directions for administering the compounds, compositions, formulations, and/or devices described herein or a combination thereof to a subject in need thereof. In some embodiments, the instructions and/or label provides direction to administer or otherwise deliver an embodiment of the implantable device described herein to a surgical site and/or wound in a subject in need thereof. In some embodiments, the instructions and/or label can specify that it is a primary surgical site and/or a revision surgical site. In some embodiments, the instructions and/or label specifies that the subject has or is at risk of developing a surgical site or wound infection. In some embodiments, the instructions and/or label specifies that the subject has or is at risk of developing a chronic and/or resistant bacterial infection. In some embodiments, the instructions and label specify that the implantable device is effective to treat and/or prevent a surgical site infection such as a chronic and/or resistant infection. In some embodiments, the instructions and label specify that the implantable device is effective to treat and/or prevent a surgical site infection caused by a bacteria. In some embodiments, the instructions and label specify that the implantable device is effective to treat and/or prevent a surgical site infection caused by a bacteria of the species Staphylococcus, Streptococcus, Pseudomonas, Enterococcus, Escherichia, Acinetobacter, or any combination thereof. In some embodiments, the instructions and label specify that the implantable device is effective to treat and/or prevent a surgical site infection caused by a bacteria of the species Staphylococcus aureus. In some embodiments, the instructions and label specify that the implantable device is effective to treat and/or prevent a surgical site infection caused by Methicillin-resistant Staphylococcus aureus (MRSA). In some embodiments, the the instructions and label specify that the implantable device is effective to treat and/or prevent biofilm formation, microbial proliferation, or both.

In some embodiments, the kit further includes one or more foreign implantable objects, soft tissue grafts, bone grafts, or any combination thereof.

### Methods of Using the Implantable Devices

The implantable devices can be delivered to a subject, such as into a surgical site during a surgical procedure, or a wound. Over time and/or in response to an external stimulus the one or more layers of the device can degrade and release the one or more therapeutic and/or preventative compounds into the environment surrounding the device. As discussed elsewhere herein, in some embodiments, the implantable device can provide extended release over time and thus treat and/or prevent infection, particularly resistant bacteria populations. It will be appreciated that in many chronic and/or resistant infections, after an initial dose of antibiotics a very small population of bacteria can remain and reemerge to perpetuate an infection and may be resistant to the original anti-infective agent used. These types of infections are a primary cause of surgical site infections, particularly those chronic cases. Current delivery devices and strategies fail in controlling and/or preventing these infections because they are incapable of delivering a therapeutic dose for a long enough period of time. Typically, these devices only deliver therapeutic does for a few days at most. This is why these are not more widely used and why systemic antibiotics are often delivered for extended periods of time prophylactically after a surgical procedure. In some cases, such as procedures on the joints and back or other areas that do not receive good blood flow, extensive wounds (such as burns), or in subjects with poor circulation to one or more areas, systemic administration of antibiotics has a limited efficacy in controlling infections in these areas and/or requires very high doses to achieve a therapeutic dose at those sites, which can lead to undesirable side effects. Further in many areas, the physical room for any implant is quite small and thus current devices simply cannot be loaded with enough active agents to deliver any therapeutic amount.

Described in exemplary embodiments herein are methods of treating and/or preventing a surgical site infection, infection at or near implanted foreign object, or wound infection in a subject in need thereof that includes implanting the implantable device as described elsewhere herein into a surgical site or other non-surgical wound in the subject in need thereof. In some embodiments, the surgical site or wound is in or near a joint of the subject, at or near an implanted foreign object in a subject, at or near the site of a skin or other soft tissue graft.

In some embodiments, the subject has or is at risk for developing a surgical, at or near an implanted foreign object in a subject, at or near the site of a skin or other soft tissue graft. In some embodiments, the subject has or is at risk for developing microbial infection, optionally a bacterial or yeast infection. In some embodiments, the bacterial infection is a chronic bacterial infection, a resistant bacterial infection, or both. In some embodiments, the bacterial infection comprises a biofilm. In some embodiments, the bacterial infection is caused by a bacteria of the species Staphylococcus, Streptococcus, Pseudomonas, Enterococcus, Escherichia, Acinetobacter, or any combination thereof. In some embodiments, the bacterial infection is caused by Staphylococcus aureus. In some embodiments, the bacterial infection is caused by Methicillin-resistant Staphylococcus aureus (MRSA).

In some embodiments, implanting occurs during a primary surgical procedure performed on the subject. In some embodiments, implanting occurs during a revision surgical procedure performed on the subject. In some embodiments, implanting occurs within 0.5-72 hours of a non-surgical wound causing trauma to the subject. Exemplary traumas include, without limitation, burns, blunt force trauma, trauma caused by impact with or by a foreign object or device, and/or the like. In some embodiments, the trauma is secondary to a disease or condition, such as diabetes or any disease that causes poor circulation in one more areas that results in e.g., a wound (e.g., ulceration, etc.). In some embodiments, the wound is a burn or other trauma requiring a skin or other soft tissue graft.

In some embodiments, the implanted foreign object is a penile implant, a soft tissue implant (e.g., breast implant, calf implant, buttock implant, cheek implant, and/or the like), replacement joint or portion thereof, fixation implant (e.g., screws, plates, and/or the like), or any combination thereof.

In some embodiments, the method includes the step of degrading one or more formable gel layers in the implant and releasing one or more therapeutic and/or preventives present in the one or more formable gel layers. Degradation can be biodegradation over time as caused by the implants response to the body of the subject. In some embodiments, degradation can be induced by exposure of the gel to an external stimuli as previously discussed.

The amount of the implantable device will vary on the specific site the device is being delivered to. In some embodiments, the amount of the gel delivered is 0.1 to 100 ug, mg, g, μL, or μg.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1—Release Kinetics of Vancomycin Loaded Gels

This Example demonstrates the evaluation of the release kinetics of antibiotics from CA (collagen-alginate) gels using ELISA with two different CA concentrations, with and without UV crosslinking. To be both novel and effective, the gels must release therapeutic doses of antibiotic over the relevant time period of 28 days.

CA gels were fabricated at two different concentrations (5% oxidized and 30 oxidized) and loaded with 33 wt. % of either vancomycin or penicillin. Further some gels were crosslinked and others were not. Gels that were crosslinked were crosslinked using UV radiation. Gels were placed in simulated body fluid and incubated at 37° C. for up to 28 days. Time course samples were tested for antibiotic concentration by ELISA and the release kinetics were calculated, which were compared to current standards of care.

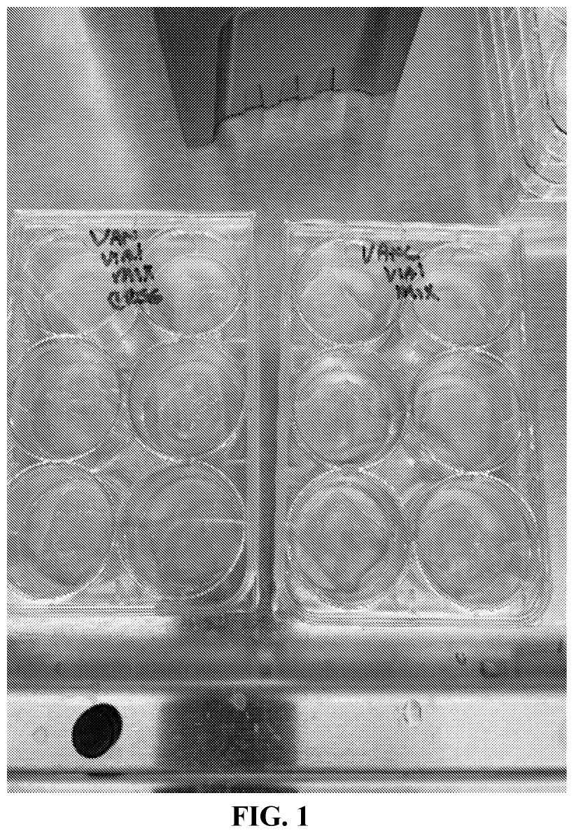

Testing of vancomycin loaded gels with the 33 wt. % concentration with and without UV radiation crosslinking was completed. As the objective was to determine the effectiveness of the gel's ability to hold the vancomycin for sustained release, the effort was simplified by looking at only one vancomycin wt. % rather than two different wt. %. PBS (Phosphate Buffered Saline) at 37° C. was used to replicate body fluid and combined the gel and PBS in a well (FIG. 1). The PBS was replaced every Tuesday and Friday during the testing and the solution was collected in 3 ml aliquots and saved at −70° C. Initial ELISA testing was completed on the collected fluids.

With respect to the gels initially, there was difficulty with the addition of the collagen to the gels with premature precipitation of the vancomycin. Therefore, the study was initiated with the alginate alone so the initial testing. Thus, the initial testing included alginate gels with vancomycin with and without crosslinking.

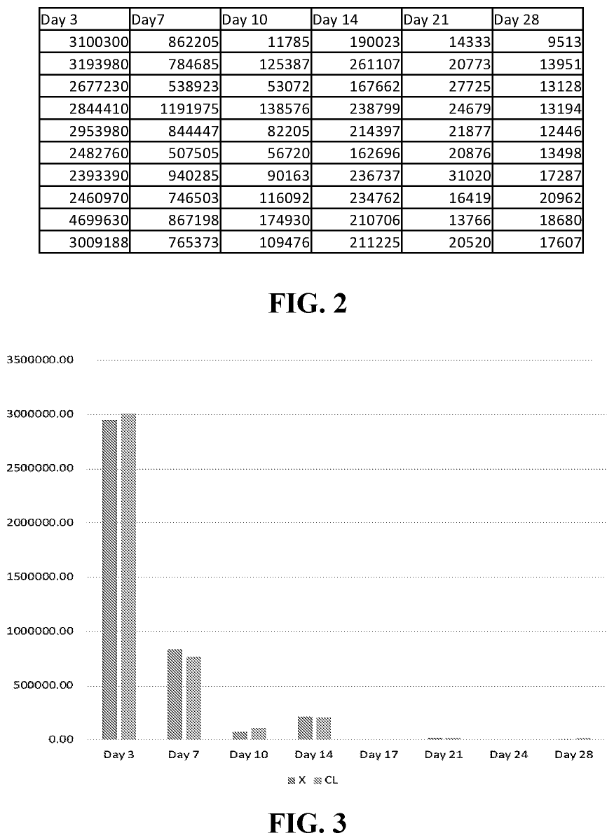

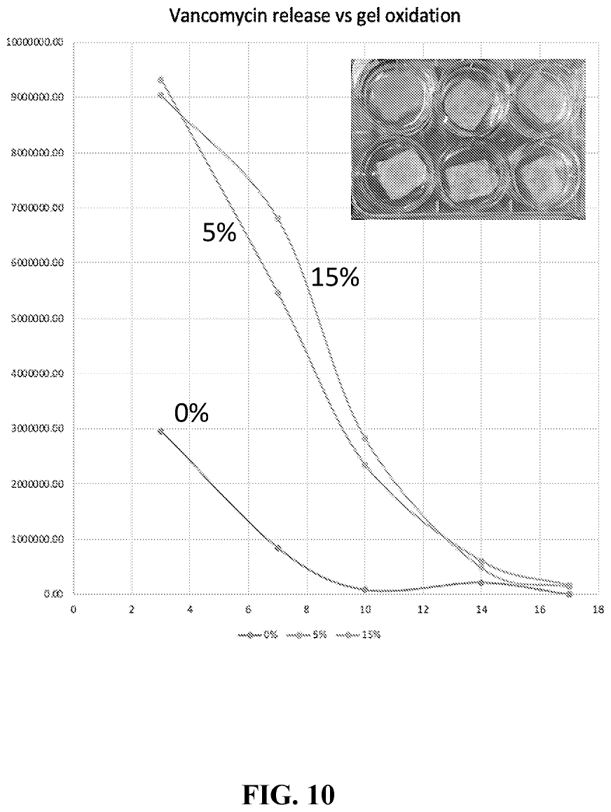

Eventually, collagen was successfully combined with the alginate to form the CA gels and the same steps as before with respect to the alginate only gels were taken to determine the elution rate of vancomycin from the collagen-alginate gels. The ELISA test was run on the following eluates listed in the and shown in FIGS. 2 and 10. Results are shown in FIGS. 1-3 and 10.

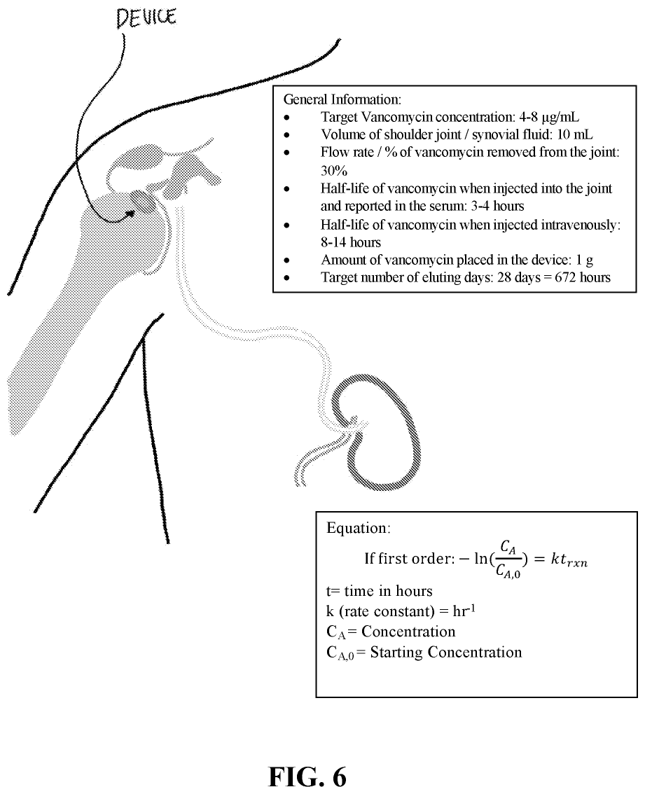

The results of the ELISA testing from FIG. 2 are graphically represented in FIG. 3 (x-axis vancomycin concentration and y axis regular and cross linked gels) and revealed that there is little difference between the elution of the antibiotic from either the cross linked and non-cross linked gels. Also, there was a high initial rate of elution of the antibiotics that tails off relatively rapidly. Without being bound by theory, it is believed that a factor affecting elution is likely related to pH. The next step in investigation was to modify the pH to reduce the rate of elution of the vancomycin. The retention of antibiotics beyond one week is a very positive finding and suggests that the device is an improvement over the existing method of directly inserting vancomycin powder. A mathematical model of the drug elution was developed and was used to predict how the gel will elute in a closed shoulder capsule (FIG. 6). It was determined that instead of releasing the vancomycin too easily the vancomycin was still being held in the gel which was the opposite of what was expected. These results of the testing indicates that release of vancomycin can be controlled release at least at day seven. Collagenase was then added to the gel to try and increase the release of antibiotics from the gel.

Next, an evaluation of the degradation kinetics of the CA gels over 28 days in vitro was completed. In brief, two different concentrations of gels with and without UV crosslinking were fabricated and tested. Gels loaded with either vancomycin or penicillin were placed in simulated body fluid with and without the addition of collagenase to mimic the in vivo environment. At 7, 14, 28, 42 days, samples were dried, weighed and the degradation rate was calculated.

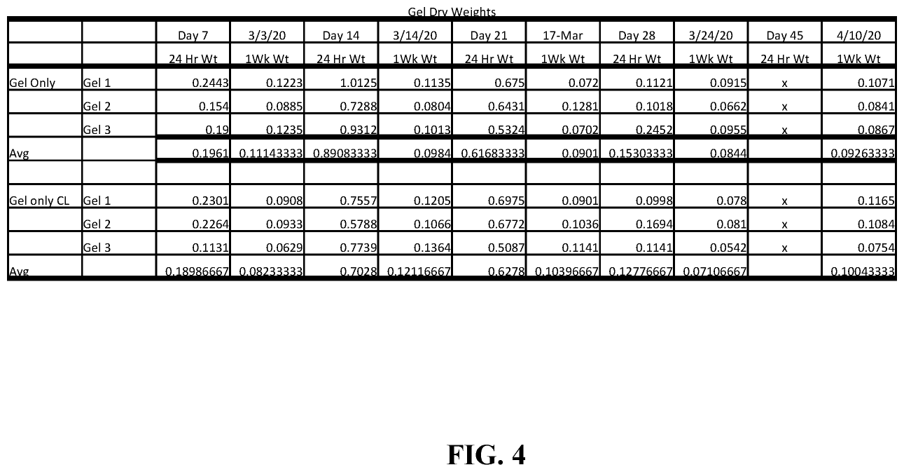

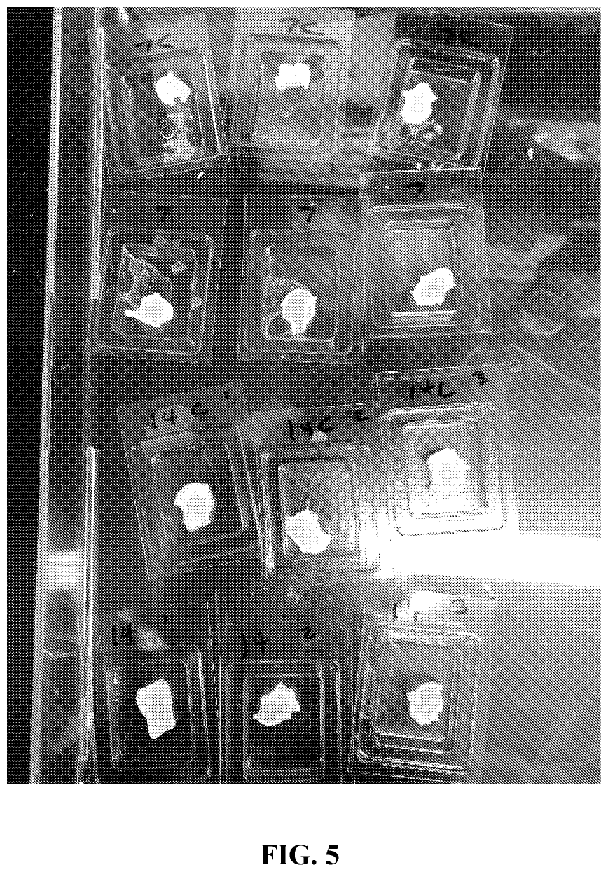

Two sets of weights for each gel was measured both immediately after removal from the PBS simulated body solution for a wet weight and then one week later after it is fully dried (FIGS. 4-5). It was observed that after 45 days the gels did not completely degrade.

### Example 2—Release of Antibiotics from Gels Over Time

This Example demonstrates results from testing of the efficacy of the eluting antibiotic over time for two concentrations of gels and two different antibiotics against cultures of Methicillin-Resistant Staphylococcus aureus (S. aureus), E. coli, and P. acnes. Similar to Example 1, gels at two different concentrations with and without crosslinking were loaded with either vancomycin or penicillin. These gels were allowed to elute in vitro for 7, 14, 21 and 28 days (as a part of the experimental process for Example 1). At the prescribed time points, gels were removed and placed in active bacteria cultures for 24 hours. After 24 hours, the gels were removed and the bacteria zone of exclusion will be measured. These data can demonstrate the efficacy of the eluting gel over time.

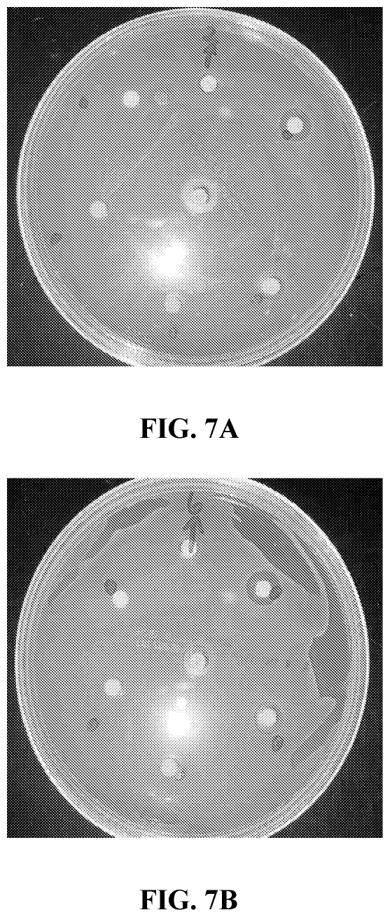

Briefly, Methicillin-Resistant Staphylococcus aureus (MRSA) was cultured in brain-heart infusion broth and diluted to match a 0.5 McFarland turbidity standard. The resulting culture was tested with gram stain to ensure the MRSA was present. After determining that the MRSA was indeed gram-positive and present, the MRSA was carefully spread onto 150 mm agar plates to inoculate the plates. 5 mcg and 30 mcg vancomycin loaded disks were placed on each agar plate as antibiotic controls. Six plates were run in this experiment, with three plates containing aliquots (from the eluates collected from the study of Example 1 Aim 1) with peroxide (FIG. 7A), while the other three plates contained aliquots run with collagenase & phosphate buffered saline (FIG. 7B).

Aliquots from days 3, 7, 14, 21, and 28 were run. 10 μL of each aliquot was pipetted onto filter paper and placed onto the agar plates. The agar plates were incubated for 24 hours at 37 degrees Celsius, and the zones of inhibition were measured the following morning. Results from the experiment can be found below in Table 1.

TABLE 1

| | Plate | 5 mcg | 30 mcg | Day 3 | Day 7 | Day 14 | Day 21 | Day 28 |
|---|---|---|---|---|---|---|---|---|
| | # | Vanc.(mm) | Vanc. (mm) | (mm) | (mm) | (mm) | (mm) | (mm) |
| Collagenase | 1 | 2 | 4 | 5 | 3 | 0 | 0 | 0 |
| & Phosphate | 2 | 1 | 5 | 4 | 3 | 1 | 0 | 0 |
| Buffered | 3 | 1 | 5 | 4 | 3 | 1 | 0 | 0 |
| Saline | | | | | | | | |
| Average | — | 1.33 | 4.67 | 4.33 | 3 | 0.67 | 0 | 0 |
| Peroxide | 4 | 2 | 5 | 5 | 3 | 2 | 0 | 0 |
| | 5 | 1 | 4 | 5 | 3 | 1 | 0 | 0 |
| | 6 | 1 | 4 | 4 | 3 | 1 | 0 | 0 |
| Average | — | 1.33 | 4.33 | 4.67 | 3 | 1.33 | 0 | 0 |

The vancomycin aliquots were observed to inhibit MRSA growth, which is highly resistant to a variety of antibiotics, and have a measurable zone of inhibition out to at least 14 days (FIGS. 7A-7B).

### Example 3—Effect of Oxidation Gel on Antibiotic Release Profile

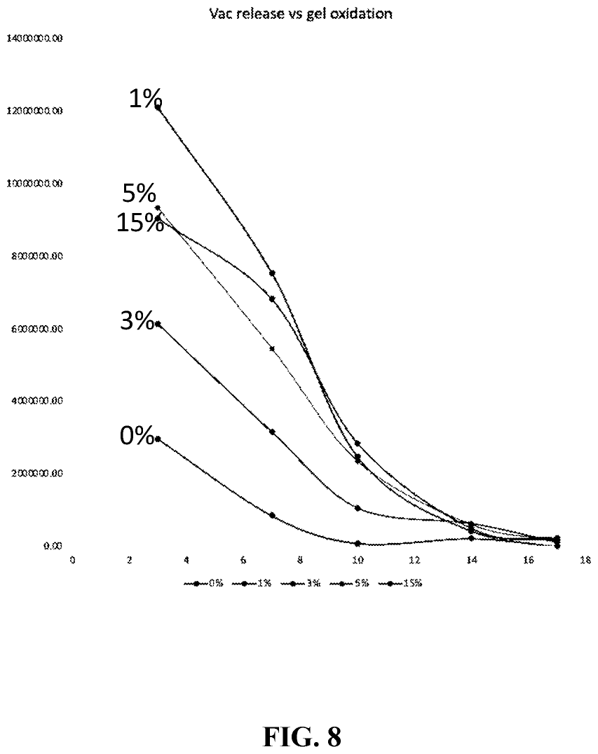

The effect of gel oxidation percent on vancomycin release was examined. The results are demonstrated in FIG. 8. To test the effect of gel oxidation percent, briefly, gels were placed in 6 well plates, and photograph of the gels at the start of test without dPBS were obtained. Five mLs of sterile PBS to each well place in 37 degrees C. CO₂ incubator. dPBS was changed 2× a week for 28 days. Photographs of gels at each collection were obtained. Collections were frozen at −80 degrees C. (1.5 mLs×2 in sterile Eppendorph tubes). Collections were acidified with 50 ul 17N HCL to resuspend any precipitate prior to the ELISA assay. Acidified collected solutions were then then tested using a standard ELISA kit assay for Vancomycin.

### Example 4—Exemplary Antibiotic Eluting Implantable Device Configurations

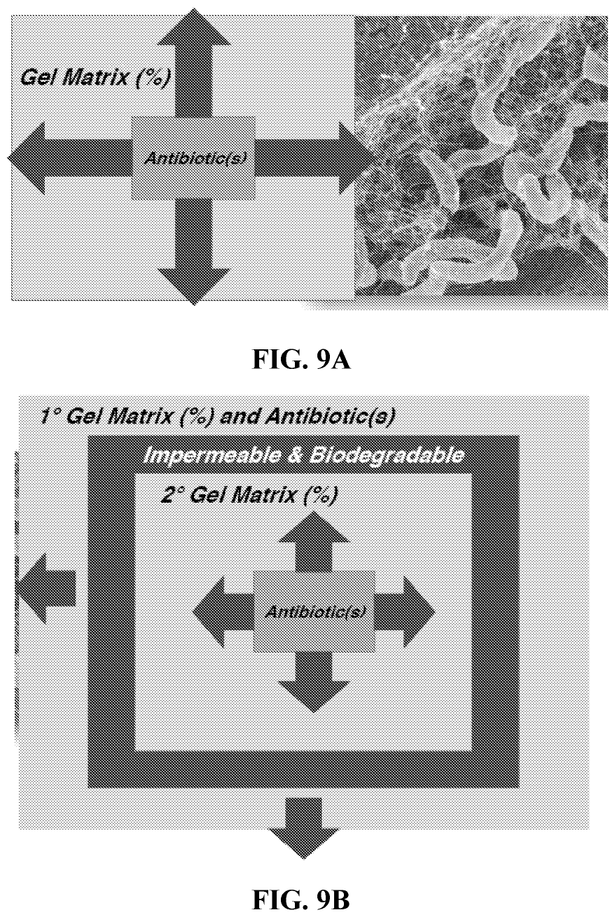

FIGS. 9A-9B show exemplary configurations of the collagen-alginate gels described herein. In some embodiments, the gel is homogenous (FIG. 9A). In some embodiments, the gel can contain 2 or more different layers of collagen-alginate-antibiotic and can optionally include one or more impermeable and degradable layers, where each impermeable and degradable layer is positioned between two of the layers and/or can exist as an outermost layer (FIG. 9B). In some examples, a first inner gel matrix can contain a different composition of collagen-alginate-antibiotic than one or more layers that are outside of it and an impermeable and biodegradable layer. Each outer layer present can contain the same or different collagen-alginate-antibiotic composition. Antibiotics are eluted from the outermost layer first. The impermeable and biodegradable layer(s) present can form a block to prevent elution from a layer before an appropriate time and/or before the elution from the outer layer(s) occurs. This configuration can allow for tuning of an elution profile and/or elution of different compounds at different times.

### Example 5—Oxidizing Alginate

Alginate was oxidized at two different concentrations (5% and 30%).

### 5% Oxidized Alginate

For the 5% oxidized alginate, about 1 gram of alginate was dissolved in about 100 mL of H₂O. About 5 mL of sodium periodate solution (C=0.05 M) was added and the solution stirred in the dark for about 24 hours to allow the oxidation reaction to occur. To end the oxidation reaction, about 0.02 mL of ethylene glycol was added. Then about 2.9 g NaCl was added to the solution and thoroughly dissolved. 200 mL of 70% ethanol was then mixed into the solution. The solution was then centrifuged for about 10 minutes at about 2000 rpm to precipitate out the alginate. The pellet was then redissolved in about 100 mL of water, after which about 200 mL of 70% ethanol was mixed in. The solution was centrifuged again. The supernatant was removed and the alginate pellets were lyophilized for about 24 hours or until completely dried.

### 30% Oxidized Alginate

For the 30% oxidized alginate, about 1 gram of alginate was dissolved in about 100 mL of H₂O. About 8 mL of sodium periodate solution (C=0.05 M) was added and the solution stirred in the dark for about 24 hours to allow the oxidation reaction to occur. To end the oxidation reaction, about 0.11 mL of ethylene glycol was added. Then about 2.9 g NaCl was added to the solution and thoroughly dissolved. 200 mL of 70% ethanol was then mixed into the solution. The solution was then centrifuged for about 10 minutes at about 2000 rpm to precipitate out the alginate. The pellet was then redissolved in about 100 ml of water, after which about 200 mL of 70% ethanol was mixed in. The solution was centrifuged again. The supernatant was removed and the alginate pellets were lyophilized for about 24 hours or until completely dried.

### Example 6—Exemplary Method of Making a Collagen Vancomycin Gel

Day 1. Prepared 4% Alginate Solution by mixing 2 grams Alginate powder (High Molecular Weight LF 20/40) in 50 mL 1× phosphate buffered saline (PBS) with a stirring until all alginate was dissolved. Placed at 4° C. overnight (about 8-16 hours) in preparation to mix with collagen. Prepared 200 mL of 0.3 M CaCl₂) in diH₂0. Prepared the collagen by thawing collagen at 4° C. overnight (about 8-16 hours). Weigh pans. Added about 1 g of collagen to each pan. Collagen was kept on ice. The weight of the pan plus the weight of the wet collagen was determined. Then the collagen was baked in the oven for a minimum of about 6 hours or overnight (about 8-16 hours).

Day 2. Prepared the Vancomycin by adding 2 ml diH₂O to a 1 g vancomycin vial and mixing on a nutator to dissolve. This took about 2 hrs. Prepared the collagen by weighing the pan+the dry collagen after baking. The protein percent was also calculated according to Table 2 below.

TABLE 2

| | Protein % | | | | |
|---|---|---|---|---|---|
| | ((pan + dry collagen) − pan)/((pan + | | | | |
| | wet collagen) − pan) * 100 = protein % | | | | |
| | | Pan + wet | Pan + dry | | |
| Pan # | Pan (g) | collagen (g) | collagen (g) | Protein % | lost |
| 1 | 0.188 | 1.057 | 0.2344 | 5.25 | 0.8226 |
| 2 | 0.1787 | 1.1271 | 0.2292 | 5.32 | 0.8979 |
| 3 | 0.209 | 1.0444 | 0.2498 | 4.88 | 0.7946 |
| 4 | 0.1745 | 1.0216 | 0.2166 | 4.97 | 0.805 |
| | | | Average | 5.11 | |
| | | | protein % | | |

The Vancomycin solution was added to the collagen at a volume needed to achieve a final weight of 100 mg Vancomycin/gel according to Equation 1 (Eq. 1), as shown in Table 3 below. The collagen was acidified down to pH 3.5 using 12 N HCl. The solution was allowed to equilibrate for at least 1 hr.

$$C 1 \times V 1 = C 2 \times V 2 \qquad ( Eq . 1 )$$

$$C 1 = initial concentration of collagen , determined from dry weight$$

$$V 1 = the volume of collagen needed ( unknown )$$

$$C 2 = known concentration of collagen ( In the present example , this was 1.5 \% )$$

$$V 2 = known volume of collagen that we need in the end ( In the present example this was 10 mL )$$

TABLE 3

| | | | | |
|---|---|---|---|---|
| V₁ = (C₂ × V₂)/C₁ | | (V₁) USE | 1.47 | mL Collagen |
| C₁ | C₂ | V₂ | | |
| 5.11 | 1.5 | 5 | | |

Total Volume (V₂) − Wanted volume (V₁) = amount of liquid needed

| | | |
|---|---|---|
| | 3.53 | mL fluid to add |
| | | to collagen |
| Add | 2 | mL Vancomycin |
| | | Solution |
| | 1.53 | mL Volume |
| | | Remaining |
| Volume HCl | 500 | μL |
| | 0.5 | mL |
| Total volume | 1.03 | mL |
| H₂O added | | |

The gels were assembled by adding the desired parts of the alginate solution to the collagen solution and mixed well. 1 mL of collagen alginate vancomycin solution into 25 mm×20 mm×5 mm were added to a cryomold. When adding the mixture to the cryomold it was ensured that the mixture covered the mold evenly by tapping the mold gently to ensure the gel covers the bottom of the mold. Filled molds were placed on a flat tray for easy handling. The filled molds were covered with 0.3 M CaCl₂) in H₂0 totaling about 1.5 mL. The gels solidified in about 15 to 30 minutes.

Collection protocol. Gels were placed in 6-well plates and gels were photographed at the start of the test without dPBS. 5 mL of sterile PBS was added to each well and the plate was incubated in a 37° C. in a CO₂ incubator. dPBS was changed two times a week for four weeks (28 days). Gels were photographed at each collection. The collections were frozen at −80° C. in appropriate collection tubes (e.g., 1.5 mL×2 in sterile Eppendorph tubes). Collections were acidified with 50 μl 17N HCL to resuspend any precipitate prior to performing an ELISA assay.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. An implantable device comprising: one or more layers of a formable gel, the formable gel of each of the one or more layers comprising: an amount of collagen; an amount of alginate; wherein the collagen and the alginate form a formable gel capable of implantation; and one or more therapeutic and/or preventative compounds dispersed throughout the formable gel, wherein the formable gel is configured to release the one or more therapeutic and/or preventative compounds over a period of time.
2. The implantable device of aspect 1, wherein the amount of collagen and/or the amount of alginate is/are effective amounts.
3. The implantable device of any one of aspects 1-2, wherein a ratio of collagen to alginate is an effective ratio.
4. The implantable device of any one of aspects 1-3, wherein the alginate, the collagen, or both are modified.
5. The implantable device of any one of aspects 1-4, wherein the alginate is at least partially oxidized.
6. The implantable device of any one of aspects 1-5, wherein the alginate is about 1-50%, about 3-30%, about 5-30%, about 7-30%, about 10-30%, about 12-30%, about 15-30%, about 20-30%, about 25-30%, about 1-25%, about 3-25%, about 5-25%, about 7-25%, about 10-25%, about 12-25%, about 15-25%, about 20-25%, about 1-20%, about 3-20%, about 5-20%, about 7-20%, about 10-20%, about 12-20%, about 15-20%, about 1-15%, about 3-15%, about 5-15%, about 7-15%, about 10-15%, about 12-15%, about 1-12%, about 1-12%, about 5-12%, about 7-12%, about 10-12%, about 1-10%, 1-3%, about 5-10% about 7-10%, about 1-7%, about 3-7%, about 5-7%, about 1-5%, about 3-5%, about 1-3% 3-50%, about 5-50%, about 7-50%, about 10-50%, about 12-50%, about 15-50%, about 20-50%, about 25-50%, about 30-50%, about 35-50%, about 40-50%, about 45-50%, about 30-45%, about 30-40%, about 30-35%, about 25-45%, about 25-40%, about 25-35%, about 20-45%, about 20-40%, about 20-35%, about 15-45%, about 15-40%, about 15-35%, about 10-45%, about 10-40%, about 10-35%, about 5-45%, about 5-40%, about 1-35%, about 1-45%, about 1-40%, or about 1-35% oxidized.
7. The implantable device of any one of aspects 1-6, wherein the period of time is at least 1-42 days or more, about 7-42 days, about 14-42 days, about 21-42 days, about 28-42 days, about 1-28 days, about 7-28 days, about 14-28 days, about 21-28 days, about 1-21 days, about 7-21 days, about 14-21 days, about 1-14 days, about 7-14 days, or about 1-7 days.
8. The implantable device of any one of aspects 1-7, wherein one or more layers of the formable gel is substantially homogenous.
9. The implantable device of any one of aspects 1-8, wherein one or more layers of the formable gel is heterogenous.
10. The implantable device of any one of aspects 1-9, wherein the implantable device further comprises one or more impermeable but degradable layers, wherein each of the one or more impermeable but degradable layers is sandwiched between two layers of the formable gel and/or is forms an external layer surrounding the one or more formable gel layers and/or one or more other degradable layers.
11. The implantable device of aspect 10, wherein one or more of the one or more impermeable but degradable layers is (a) biodegradable, (b) degradable in response to an external stimuli, or both (a) and (b).
12. The implantable device of any one of aspects 1-11, wherein one or more therapeutic and/or preventative compounds are each present in one or more of the one or more layers of the formable gel at 1-70 wt. % of the formable gel layer, at 1-65% of the formable gel layer, 1-60% of the formable gel layer, 1-55% of the formable gel layer, 1-50% of the formable gel layer, 1-45 wt. % of the formable gel layer, 1-40 wt. % of the formable gel layer, 1-35 wt. % of the formable gel layer, 1-30 wt. % of the formable gel layer, 1-25 wt. % of the formable gel layer, 1-20 wt. % of the formable gel layer, 1-15 wt. % of the formable gel layer, 1-10 wt. % of the formable gel layer, or 1-5 wt. % of the formable gel layer.
13. The implantable device of any one of aspects 1-12, wherein the one or more therapeutic and/or preventative compounds are selected from the group consisting of: an anti-infective, a chemotherapeutic, an immunomodulator, an antipyretic, an analgesic, an antispasmodic, an anti-inflammatory, an anti-histamine, a radiation sensitizer, a chemotherapeutic sensitizer, an anti-infective sensitizer, a biologic factor, a bisphosphonate, or any combination thereof.
14. The implantable device of any one of aspects 1-13, wherein the one or more therapeutic and/or preventative compounds comprises an anti-infective agent.
15. The implantable device of aspect 14, wherein the one or more therapeutic and/or preventative compounds comprise an antibiotic.
16. The implantable device of any one of aspects 1-15, wherein the one or more therapeutic and/or preventative compounds comprises an aminoglycoside or a derivative thereof.
17. The implantable device of aspect 16, wherein the aminoglycoside is selected from paromomycin, tobramycin, gentamicin, amikacin, kanamycin, neomycin, and any combination thereof.
18. The implantable device of any one of aspects 1-17, wherein the one or more therapeutic and/or preventative compounds are each independently selected from an aminoglycoside or a derivative thereof, a carbapenem or a derivative thereof, a cephalosporin or a derivative thereof, a glycopeptide or a derivative thereof, a glycylcycline or a derivative thereof, a lincomycin or a derivative thereof, a macrolide or a derivative thereof, a penicillin or a derivative thereof, a quinolone or a derivative thereof, a sulfonamide or a derivative thereof, a tetracycline or a derivative thereof, or any combination thereof.
19. The implantable device of aspect 18, wherein (a) the aminoglycoside comprises paromomycin, tobramycin, gentamicin, amikacin, kanamycin, and neomycin or any combination thereof; (b) the carbapenem comprises doripenem, meropenem, ertapenem, and cilastatin/imipenem; (c) the cephalosporin comprises cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, ceftazidime, or any combination thereof; (d) the glycopeptide comprises vancomycin, dalbavancin, oritavancin, telvancin, or any combination thereof; (e) the glycylcycline comprises tigecycline; (f) the lincomycin comprises clindamycin or lincomycin; (g) the macrolide comprises telithromycin, fidaxomicin, erythromycin, azithromycin, clarithromycin, dirithromycin, troleandomycin, linezolid, sulfamethoxazole/trimethoprim, rifaximin, chloramphenicol, fosfomycin, metronidazole, aztreonam, bacitracin, or any combination thereof; (h) the penicillin comprises amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, nafcillin, or any combination thereof; (i) the quinolone comprises lomefloxacin, norfloxacin, ofloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, sparfloxacin, or any combination thereof; (j) the sulfonamide comprises sulfamethoxazole/trimethoprim, sulfasalazine, sulfasoxazole, or any combination thereof; (k) the tetracycline (e.g. doxycycline, demeclocycline, minocycline, doxycycline/salicylic acid, doxycycline/omega-3 polyunsaturated fatty acids, and tetracycline; or (1) any combination of (a)-(k).
20. The implantable device of any one aspects 1-19, wherein the implantable device is effective to treat and/or prevent microbial infection, microbial proliferation, biofilm formation, or any combination thereof.
21. A kit comprising: the implantable device of any one of aspects 1-20.
22. A method of treating and/or preventing a surgical site infection, infection at or near implanted foreign object, or wound infection in a subject in need thereof, the method comprising: implanting the implantable device of any one of aspects 1-20 into a surgical site or wound in the subject in need thereof.
23. The method of aspect 22, wherein the surgical site or wound is in or near a joint of the subject, at or near an implanted foreign object in a subject, at or near the site of a skin or other soft tissue graft.
24. The method of any one of aspects 22-23, wherein the subject has or is at risk for developing a surgical site, implant foreign object site, or wound site infection.
25. The method of any one of aspects 22-24, wherein the subject has or is at risk for developing a microbial infection.
26. The method of aspect 25, wherein the microbial infection comprises a bacterial infection, a yeast infection, or both.
27. The method of aspect 26, wherein the bacterial infection is a chronic bacterial infection, a resistant bacterial infection, or both.
28. The method of any one of aspects 26-27, wherein the bacterial infection comprises a biofilm.
29. The method of any one of aspects 26-28, wherein the bacterial infection is caused by a bacteria of the species Staphylococcus, Streptococcus, Pseudomonas, Enterococcus, Escherichia, Acinetobacter, or any combination thereof.
30. The method of aspect 29, wherein the bacterial infection is caused by Staphylococcus aureus.
31. The method of any one of aspects 22-30, wherein implanting occurs during a primary surgical procedure performed on the subject.
32. The method of any one of aspects 22-31, wherein implanting occurs during a revision surgical procedure performed on the subject.
33. The method of any one of aspects 22-32, wherein implanting occurs within 0.5-72 hours of a non-surgical wound causing trauma to the subject.
34. The method of any one of aspects 22-33, wherein the wound is a burn or other trauma requiring a skin graft.
35. The method of any one of aspects 22-34, wherein the implanted foreign object is a penile implant, a soft tissue implant, a replacement joint or portion thereof, a fixation implant, or any combination thereof.
36. The method of aspect 35, wherein (a) the soft tissue implant comprises a breast implant, calf implant, buttock implant, or a cheek implant; (b) the fixation implant comprises a screw, a plate, a cage, a rod, a pin, an anchor, a disc, or any combination thereof; or (c) both (a) and (b).

What is claimed is:

1. An implantable device comprising:
one or more layers of a formable gel, the formable gel of each of the one or more layers comprising:
an amount of collagen;
an amount of alginate; wherein the collagen and the alginate form a formable gel capable of implantation; and
one or more therapeutic and/or preventative compounds dispersed throughout the formable gel, wherein the formable gel is configured to release the one or more therapeutic and/or preventative compounds over a period of time.

2. The implantable device of claim 1, wherein the amount of collagen and/or the amount of alginate is/are effective amounts.

3. The implantable device of claim 1, wherein a ratio of collagen to alginate is an effective ratio.

4. The implantable device of claim 1, wherein the alginate, the collagen, or both are modified.

5. The implantable device of claim 1, wherein the alginate is at least partially oxidized.

6. The implantable device of claim 1, wherein the alginate is about 1-50%, about 3-30%, about 5-30%, about 7-30%, about 10-30%, about 12-30%, about 15-30%, about 20-30%, about 25-30%, about 1-25%, about 3-25%, about 5-25%, about 7-25%, about 10-25%, about 12-25%, about 15-25%, about 20-25%, about 1-20%, about 3-20%, about 5-20%, about 7-20%, about 10-20%, about 12-20%, about 15-20%, about 1-15%, about 3-15%, about 5-15%, about 7-15%, about 10-15%, about 12-15%, about 1-12%, about 1-12%, about 5-12%, about 7-12%, about 10-12%, about 1-10%, 1-3%, about 5-10%, about 7-10%, about 1-7%, about 3-7%, about 5-7%, about 1-5%, about 3-5%, about 1-3% 3-50%, about 5-50%, about 7-50%, about 10-50%, about 12-50%, about 15-50%, about 20-50%, about 25-50%, about 30-50%, about 35-50%, about 40-50%, about 45-50%, about 30-45%, about 30-40%, about 30-35%, about 25-45%, about 25-40%, about 25-35%, about 20-45%, about 20-40%, about 20-35%, about 15-45%, about 15-40%, about 15-35%, about 10-45%, about 10-40%, about 10-35%, about 5-45%, about 5-40%, about 1-35%, about 1-45%, about 1-40%, or about 1-35% oxidized.

7. The implantable device of claim 1, wherein the period of time is at least 1-42 days or more, about 7-42 days, about 14-42 days, about 21-42 days, about 28-42 days, about 1-28 days, about 7-28 days, about 14-28 days, about 21-28 days, about 1-21 days, about 7-21 days, about 14-21 days, about 1-14 days, about 7-14 days, or about 1-7 days.

8. The implantable device of claim 1, wherein one or more layers of the formable gel is substantially homogenous.

9. The implantable device of claim 1, wherein one or more layers of the formable gel is heterogenous.

10. The implantable device of claim 1, wherein the implantable device further comprises one or more impermeable but degradable layers, wherein each of the one or more impermeable but degradable layers is sandwiched between two layers of the formable gel and/or is forms an external layer surrounding the one or more formable gel layers and/or one or more other degradable layers.

11. The implantable device of claim 10, wherein one or more of the one or more impermeable but degradable layers is (a) biodegradable, (b) degradable in response to an external stimuli, or both (a) and (b).

12. The implantable device of claim 1, wherein one or more therapeutic and/or preventative compounds are each present in one or more of the one or more layers of the formable gel at 1-70 wt. % of the formable gel layer, at 1-65% of the formable gel layer, 1-60% of the formable gel layer, 1-55% of the formable gel layer, 1-50% of the formable gel layer, 1-45 wt. % of the formable gel layer, 1-40 wt. % of the formable gel layer, 1-35 wt. % of the formable gel layer, 1-30 wt. % of the formable gel layer, 1-25 wt. % of the formable gel layer, 1-20 wt. % of the formable gel layer, 1-15 wt. % of the formable gel layer, 1-10 wt. % of the formable gel layer, or 1-5 wt. % of the formable gel layer.

13. The implantable device of claim 1, wherein the one or more therapeutic and/or preventative compounds are selected from the group consisting of: an anti-infective, a chemotherapeutic, an immunomodulator, an antipyretic, an analgesic, an antispasmodic, an anti-inflammatory, an anti-histamine, a radiation sensitizer, a chemotherapeutic sensitizer, an anti-infective sensitizer, a biologic factor, a bisphosphonate, or any combination thereof.

14. The implantable device of claim 1, wherein the one or more therapeutic and/or preventative compounds comprises an anti-infective agent.

15. The implantable device of claim 14, wherein the one or more therapeutic and/or preventative compounds comprise an antibiotic.

16. The implantable device of claim 1, wherein the one or more therapeutic and/or preventative compounds comprises an aminoglycoside or a derivative thereof.

17. The implantable device of claim 16, wherein the aminoglycoside is selected from paromomycin, tobramycin, gentamicin, amikacin, kanamycin, neomycin, and any combination thereof.

18. The implantable device of claim 1, wherein the one or more therapeutic and/or preventative compounds are each independently selected from an aminoglycoside or a derivative thereof, a carbapenem or a derivative thereof, a cephalosporin or a derivative thereof, a glycopeptide or a derivative thereof, a glycylcycline or a derivative thereof, a lincomycin or a derivative thereof, a macrolide or a derivative thereof, a penicillin or a derivative thereof, a quinolone or a derivative thereof, a sulfonamide or a derivative thereof, a tetracycline or a derivative thereof, or any combination thereof.

19. The implantable device of claim 18, wherein
(a) the aminoglycoside comprises paromomycin, tobramycin, gentamicin, amikacin, kanamycin, and neomycin or any combination thereof;
(b) the carbapenem comprises doripenem, meropenem, ertapenem, and cilastatin/imipenem;
(c) the cephalosporin comprises cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, ceftazidime, or any combination thereof;
(d) the glycopeptide comprises vancomycin, dalbavancin, oritavancin, telvancin, or any combination thereof;
(e) the glycylcycline comprises tigecycline;
(f) the lincomycin comprises clindamycin or lincomycin;
(g) the macrolide comprises telithromycin, fidaxomicin, erythromycin, azithromycin, clarithromycin, dirithromycin, troleandomycin, linezolid, sulfamethoxazole/trimethoprim, rifaximin, chloramphenicol, fosfomycin, metronidazole, aztreonam, bacitracin, or any combination thereof;
(h) the penicillin comprises amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, nafcillin, or any combination thereof;
(i) the quinolone comprises lomefloxacin, norfloxacin, ofloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, sparfloxacin, or any combination thereof;
(j) the sulfonamide comprises sulfamethoxazole/trimethoprim, sulfasalazine, sulfasoxazole, or any combination thereof;
(k) the tetracycline (e.g. doxycycline, demeclocycline, minocycline, doxycycline/salicyclic acid, doxycycline/omega-3 polyunsaturated fatty acids, and tetracycline; or
(l) any combination of (a)-(k).

20. The implantable device of claim 1, wherein the implantable device is effective to treat and/or prevent microbial infection, microbial proliferation, biofilm formation, or any combination thereof.

21. A kit comprising:
the implantable device of any one of claims 1-20.

22. A method of treating and/or preventing a surgical site infection, infection at or near implanted foreign object, or wound infection in a subject in need thereof, the method comprising:
implanting the implantable device of any one of claims 1-20 into a surgical site or wound in the subject in need thereof.

23. The method of claim 22, wherein the surgical site or wound is in or near a joint of the subject, at or near an implanted foreign object in a subject, at or near the site of a skin or other soft tissue graft.

24. The method of claim 22, wherein the subject has or is at risk for developing a surgical site, implant foreign object site, or wound site infection.

25. The method of claim 22, wherein the subject has or is at risk for developing a microbial infection.

26. The method of claim 25, wherein the microbial infection comprises a bacterial infection, a yeast infection, or both.

27. The method of claim 26, wherein the bacterial infection is a chronic bacterial infection, a resistant bacterial infection, or both.

28. The method of claim 26, wherein the bacterial infection comprises a biofilm.

29. The method of claim 26, wherein the bacterial infection is caused by a bacteria of the species Staphylococcus, Streptococcus, Pseudomonas, Enterococcus, Escherichia, Acinetobacter, or any combination thereof.

30. The method of claim 29, wherein the bacterial infection is caused by Staphylococcus aureus.

31. The method of claim 22, wherein implanting occurs during a primary surgical procedure performed on the subject.

32. The method of claim 22, wherein implanting occurs during a revision surgical procedure performed on the subject.

33. The method of claim 22, wherein implanting occurs within 0.5-72 hours of a non-surgical wound causing trauma to the subject.

34. The method of claim 22, wherein the wound is a burn or other trauma requiring a skin graft.

35. The method of claim 22, wherein the implanted foreign object is a penile implant, a soft tissue implant, a replacement joint or portion thereof, a fixation implant, or any combination thereof.

36. The method of claim 35, wherein
(a) the soft tissue implant comprises a breast implant, calf implant, buttock implant, or a cheek implant;
(b) the fixation implant comprises a screw, a plate, a cage, a rod, a pin, an anchor, a disc, or any combination thereof; or
(c) both (a) and (b).
